(19) United States
(12) Patent Application Publication
(10) Pub. No.: US 2005/0267081 A1
(43) Pub. Date: Dec. 1, 2005

(54) Use of a combination of ethinyloestradiol and chlormadinone acetate to produce a pharmaceutical preparation

(76) Inventors: Janine Klose, Wurselen (DE); Johannes Bartholomaus, Aachen (DE); Klaus-Michael Wilsmann, Roetgen (DE); Georg Schramm, Stolberg (DE)

Correspondence Address: PERMAN & GREEN, 425 POST ROAD, FAIRFIELD, CT, 06824, US

(21) Appl. No.: 11/009,361

(22) Filed: Dec. 10, 2004

(30) Foreign Application Priority Data
May 28, 2004 (DE) 10 2004 026 669.7

(51) Int. Cl. A61K 31/56

(52) U.S. Cl. 514/170

(57) ABSTRACT

The invention relates to the use of a combination of ethinyloestradiol and chlormadinone acetate to produce a pharmaceutical preparation for the treatment of androgen-induced disorders and for hormone replacement therapy and for the treatment of dysmenorrhoea and for stabilising the menstrual cycle and for the treatment of menstrual cycle-dependent complaints and for contraception simultaneously in women.

The present invention relates to the use of a combination of ethinyloestradiol and chlormadinone acetate to produce a pharmaceutical preparation
for the treatment of androgen-induced disorders and
for hormone replacement therapy and
for the treatment of dysmenorrhoea and
for stabilising the menstrual cycle and
for the treatment of menstrual cycle-dependent complaints and
for contraception
   simultaneously in women.

Many women suffer from androgen-induced disorders, such as acne, hirsutism (e.g. unwanted facial hair), androgenetic alopecia and/or seborrhoea, in particular as they grow older.

A large number of women suffer from dysmenorrhoea and/or menstrual cycle-dependent complaints, in particular premenstrual syndrome, such as headaches and/or migraine.

In women of over 35 in particular, especially during the pre- and perimenopause, hormonal irregularities, such as irregular menstrual cycles, and vasomotor disorders, such as hot flushes, sweating and insomnia, may occur.

For these and other reasons, hormone replacement therapy is often necessary. At the same time, women, in particular women over 35, want reliable contraceptive protection.

Effective combatting of dysmenorrhoea and in particular of premenstrual syndrome is also necessary.

A description has already been given in EP-A-0 398 460 of a combined hormone preparation for treating some of the above-mentioned complaints, in particular during the pre- or perimenopause and including reliable contraceptive protection.

This preparation is suitable for women with high blood pressure, since the gestagen component counteracts it.

However, since many women suffer from the above-listed complaints, in particular in the pre- and perimenopause, without having high blood pressure, there is a need for a pharmaceutical preparation which, in addition to its excellent contraceptive action, is also suitable simultaneously for the treatment of androgen-induced disorders, for hormone replacement therapy, for the treatment of dysmenorrhoea and for the treatment of menstrual cycle-dependent complaints.

This object is achieved by the use of a combination of ethinyloestradiol and chlormadinone acetate to produce a pharmaceutical preparation
for the treatment of androgen-induced disorders and
for hormone replacement therapy and
for the treatment of dysmenorrhoea and
for stabilising the menstrual cycle and
for the treatment of menstrual cycle-dependent complaints and
for contraception
   simultaneously in women.

To produce the pharmaceutical preparation, a combination of 5 to 50 μg, preferably 5 to 30 μg, of ethinyloestradiol and 1 to 5 mg of chlormadinone acetate is preferably used. A combination of 15 μg, 20 μg or 30 μg of ethinyloestradiol and 1 mg, 2, 3, 4 or 5 mg of chlormadinone acetate is particularly preferred.

By using the stated combination to produce a pharmaceutical preparation, it is possible not only to achieve excellent contraceptive action but also simultaneously to treat androgen-induced disorders, such as for example acne, hirsutism, androgenetic alopecia and/or seborrhoea, to provide hormone replacement therapy, in particular to combat vasomotor complaints, in particular in the pre- and perimenopause, such as hot flushes, sweating and/or insomnia and to treat or stabilise irregular menstrual cycles, as well as to combat dysmenorrhoea and menstruation-dependent complaints, in particular premenstrual syndrome often associated with headaches and/or migraine.

Surprisingly, the gestagen chlormadinone acetate used in the hormone combination has proven particularly suitable for combatting menstruation-dependent complaints, in particular premenstrual syndrome and the associated headaches and/or migraine and for the treatment of dysmenorrhoea.

The pharmaceutical preparation produced using a combination of ethinyloestradiol and chlormadinone acetate is also particularly suitable for treating women over 35, preferably women in the pre- and perimenopause, due to the above-stated broad range of efficacy.

The pharmaceutical preparation produced according to the invention is preferably formulated in the form of tablets. To this end, it is provided in particular in the form of at least 21 hormone-containing daily units, which are intended for uninterrupted administration, optionally in combination with 7 to 3 hormone-free daily units.

To treat the above-listed complaints or disorders, the pharmaceutical preparation may also be provided in the form of hormone-containing daily units for uninterrupted administration over several years, preferably up to 2 years, particularly preferably up to 1 year, optionally in combination with 7 to 3 hormone-free daily units.

However, the pharmaceutical preparation according to the invention may also be prepared in a dosage form with fewer than 365 hormone-containing daily units, such as for example with 77 to 193 or 42 to 52 hormone-containing daily units for uninterrupted administration, optionally in combination with 7 to 3 hormone-free daily units.

Instead of the 7 to 3 hormone-free daily units, an interval in taking of corresponding length may also be provided before taking a further package of formulated pharmaceutical preparations. Therefore, the oral dosage form with the above-listed hormone-containing daily units optionally in combination with hormone-free daily units may also take the form of a kit, which contains a plurality of such dosage forms for continued administration, interrupted by hormone-free daily units or a corresponding interval in taking.

Each of the hormone-containing daily units preferably comprises the same amount of ethinyloestradiol or of chlormadinone acetate.

In a further preferred embodiment, the hormone-containing daily units may vary in a known way in their content of ethinyloestradiol or of chlormadinone acetate according to a biphasic or triphasic tablet-taking cycle over 21 to 25 days.

Processes for the production of a pharmaceutical preparation comprising the hormone combination of ethinyloestradiol and chlormadinone acetate and corresponding formulation processes for the production of a dosage form, in particular an oral dosage form in the form of tablets, are known to the person skilled in the art.

### EXAMPLES

### Example 1

Composition

| | Per tablet | |
|---|---|---|
| Ethinyloestradiol | 0.020 | mg |
| Chlormadinone acetate | 2.000 | mg |
| Povidone K30 | 3.000 | mg |
| Lactose | 31.980 | mg |
| Maize starch | 12.000 | mg |
| Magnesium stearate | 0.500 | mg |
| Highly disperse silicon dioxide | 0.500 | mg |

Ethinyloestradiol (EE) and povidone K30 (polyvinylpyrrolidone, PVP) were dissolved in 600 ml of ethanol. Chlormadinone acetate (particle size 90%<50 μm), lactose and maize starch were mixed in a mixer/pelletiser (Diosna P25) for 5 mins and then moistened thoroughly and mixed with the ethanolic EE/PVP solution. The moist composition was forced through a 3 mm screen and dried in a vacuum drying cabinet. The dried granular product was disagglomerated through a 0.6 mm screen, mixed with magnesium stearate and highly disperse silicon dioxide and pressed on a tablet press with 5 mm punches into tablets with a weight of 50 mg. The tablets were coated with a methylhydroxypropylcellulose-based coating (e.g. Opadry YS-1-2184); coating composition 2 mg per tablet

### Example 2

Composition

| | Per tablet | |
|---|---|---|
| Ethinyloestradiol | 0.015 | mg |
| Chlormadinone acetate | 1.000 | mg |
| Povidone K30 | 3.000 | mg |
| Lactose | 32.985 | mg |
| Maize starch | 12.000 | mg |
| Magnesium stearate | 0.500 | mg |
| Highly disperse silicon dioxide | 0.500 | mg |

Ethinyloestradiol (EE) and povidone K30 (PVP) were dissolved in 600 ml of ethanol. Chlormadinone acetate (particle size 90%<50 μm), lactose and maize starch were mixed in a mixer/pelletiser (Diosna P25) for 5 mins and then moistened thoroughly and mixed with the ethanolic EE/PVP solution. The moist composition was forced through a 3 mm screen and dried in a vacuum drying cabinet. The dried granular product was disagglomerated through a 0.6 mm screen, mixed with magnesium stearate and highly disperse silicon dioxide and pressed on a tablet press with 5 mm punches into tablets with a weight of 50 mg.

The tablets were coated with a methylhydroxypropylcellulose-based coating of the following composition (coating composition 2 mg per tablet)

| | | |
|---|---|---|
| Methylhydroxypropylcellulose 6 mPa · s, | 0.1351 | kg |
| Polyethylene glycol 6000 | 0.0395 | kg |
| Propylene glycol | 0.0054 | kg |
| Purified water | 1.6200 | kg |

### Example 3

Composition

| | Per tablet | |
|---|---|---|
| Ethinyloestradiol | 0.015 | mg |
| Chlormadinone acetate | 2.000 | mg |
| Povidone K30 | 4.000 | mg |
| Lactose | 634.85 | mg |
| Maize starch | 10.000 | mg |
| Magnesium stearate | 0.500 | mg |

Ethinyloestradiol (EE) and povidone K30 (PVP) were dissolved in 950 ml of ethanol. Chlormadinone acetate (particle size 90%<50 μm), lactose and maize starch were mixed in a mixer/pelletiser (Diosna P25) for 5 mins and then moistened thoroughly and mixed with the ethanolic EE/PVP solution. The moist composition was forced through a 3 mm screen and dried in a vacuum drying cabinet. The dried granular product was disagglomerated through a 0.6 mm screen, mixed with magnesium stearate and pressed on a tablet press with 6 mm punches into tablets with a weight of 80 mg.

The tablets were coated with a methylhydroxypropylcellulose-based coating of the following composition (coating composition 2 mg per tablet)

| | | |
|---|---|---|
| Methylhydroxypropylcellulose 6 mPa · s, | 0.1351 | kg |
| Polyethylene glycol 6000 | 0.0395 | kg |
| Propylene glycol | 0.0054 | kg |
| Purified water | 1.6200 | kg |

### Example 4

Composition

| | Per tablet | |
|---|---|---|
| Ethinyloestradiol | 0.030 | mg |
| Chlormadinone acetate | 5.000 | mg |
| Povidone K30 | 4.500 | mg |
| Lactose | 60.470 | mg |
| Maize starch | 10.000 | mg |
| Magnesium stearate | 0.500 | mg |

Ethinyloestradiol (EE) and povidone K30 (PVP) were dissolved in 950 ml of ethanol. Chlormadinone acetate (particle size 90%<50 μm), lactose and maize starch were mixed in a mixer/pelletiser (Diosna P25) for 5 mins and then moistened thoroughly and mixed with the ethanolic EE/PVP solution. The moist composition was forced through a 3 mm screen and dried in a vacuum drying cabinet. The dried granular product was disagglomerated through a 0.6 mm screen, mixed with magnesium stearate and pressed on a tablet press with 6 mm punches into tablets with a weight of 80 mg.

The tablets were coated with a methylhydroxypropylcellulose-based coating of the following composition (coating composition 1 mg per tablet)

| | | |
|---|---|---|
| Methylhydroxypropylcellulose 6 mPa · s, | 0.068 | kg |
| Polyethylene glycol 6000 | 0.020 | kg |
| Propylene glycol | 0.002 | kg |
| Purified water | 0.810 | kg |

1. Use of a combination of ethinyloestradiol and chlormadinone acetate to produce a pharmaceutical preparation
for the treatment of androgen-induced disorders and
for hormone replacement therapy and
for the treatment of dysmenorrhoea and
for stabilising the menstrual cycle and
for the treatment of menstrual cycle-dependent complaints and
for contraception
simultaneously in women.

2. Use according to claim 1 for administration to women over 35, preferably women in the pre- or perimenopause.

3. Use according to claim 1, characterised in that the combination consists of 5 to 50 μg of ethinyloestradiol and 1 to 5 mg of chlormadinone acetate.

4. Use according to claim 1, characterised in that the combination consists of 5 to 30 μg of ethinyloestradiol and 1 to 5 mg of chlormadinone acetate.

5. Use according to claim 4, characterised in that the combination consists of 15 μg, 20 μg or 30 μg of ethinyloestradiol and 1, 2, 3, 4 or 5 mg of chlormadinone acetate.

6. Use according to claim 1, characterised in that the pharmaceutical preparation is prepared in the form of at least 21 hormone-containing daily units for uninterrupted administration, optionally in combination with 7 to 3 hormone-free daily units.

7. Use according to claim 6, characterised in that the pharmaceutical preparation is prepared in the form of hormone-containing daily units for uninterrupted administration over several years, preferably up to 2 years, particularly preferably up to 1 year, optionally in combination with 7 to 3 hormone-free daily units.

8. Use according to claim 6, characterised in that the pharmaceutical preparation is prepared in the form of 77 to 193 hormone-containing daily units optionally in combination with 7 to 3 hormone-free daily units.

9. Use according to claim 6, characterised in that the pharmaceutical preparation is prepared in the form of 42 to 52 hormone-containing daily units optionally in combination with 7 to 3 hormone-free daily units.

10. Use according to claim 6, characterised in that the pharmaceutical preparation is prepared in the form of 21 to 25 hormone-containing daily units optionally in combination with 7 to 3 hormone-free daily units.

11. Use according to claim 1, characterised in that, with regard to quantities, the pharmaceutical preparation comprises in each of the hormone-containing daily units the same combination of ethinyloestradiol and chlormadinone acetate.

12. Use according to claim 1 for the treatment of the androgen-induced disorders acne, hirsutism, androgenetic alopecia, seborrhoea.

13. Use according to claim 1 for hormone replacement therapy to treat vasomotor disorders, in particular to treat hot flushes, sweating, insomnia.

14. Use according to claim 1 for the treatment of the menstrual cycle-dependent complaint premenstrual syndrome, in particular headache and/or migraine.
